(19) United States
(12) Patent Application Publication
(10) Pub. No.: US 2008/0260833 A1
(43) Pub. Date: Oct. 23, 2008

(54) Drug delivery vehicle containing vesicles in a hydrogel base

(76) Inventors: Thomas Hirt, Rebstein (CH); Wolfgang Meier, Therwil (CH); Zhihua Lu, Johns Creek, GA (US); Xianbo Hu, Alpharetta, GA (US)

Correspondence Address: LAW OFFICE OF COLLEN A. BEARD, LLC, P. O. BOX 1064, DECATUR, GA, 30031-1064, US

(21) Appl. No.: 12/148,407

(22) Filed: Apr. 18, 2008

Related U.S. Application Data
(60) Provisional application No. 60/925,529, filed on Apr. 20, 2007.

(51) Int. Cl. A61K 9/10

(52) U.S. Cl. 424/486

(57) ABSTRACT

A drug delivery vehicle having active agent loaded vesicles in a hydrogel matrix; desirably either or both of the vesicles and matrix are made of at least one stimulus responsive polymer so that active agent is released in response to contact with a stimulus.

### RELATED APPLICATION

The present application is related to and claims priority to U. S. Provisional Application Ser. No. 60/925,529 filed Apr. 20, 2007, the entire contents of which are incorporated herein by reference.

### BACKGROUND OF THE INVENTION

The invention is related to drug delivery and more specifically related to vehicles for drug delivery which comprise drug loaded vesicles in a hydrogel matrix.

According to the present invention, an active agent is encapsulated in vesicles which are entrapped in a hydrogel matrix. Desirably the vesicles are made at least partially of a stimulus responsive polymer so that release of the active agent from the vesicles, and the vehicle, is triggered by exposure to the stimulus. Drug release from the vehicle can be programmed through design of the vesicles and the matrix. Drug release from the vesicles and the matrix can also be through passive release.

Research in the field of drug delivery is ongoing; the aim is to design methods and devices to deliver active agents to the human body. There are many factors to be considered in designing a method for delivery of a particular active agent. In some cases a particular drug release profile is desired, or the rate of delivery of the drug may be important and may determine the delivery means. In other cases, the characteristics of the drug determine at least in part how it is formulated. For example, the drug may be easily broken down by the body, or in a particular region of the body, and it may be desirable to protect the drug until it can reach its intended target.

Liposomes, spherical vesicles with a membrane composed of a phospholipid and cholesterol bilayer, have been explored as agents for protection and delivery of active agents. Encapsulation of a bioactive agent in a liposome can provide for a more prolonged release of the agent because the liposome membrane can be prepared or modified to retard the leak of the agent. Liposomes can also protect a drug from degradation in some cases. The use of liposomes for drug delivery is taught, for example, in Rahman et al., U.S. Pat. No. 3,993,754; Sears, U.S. Pat. No. 4,145,410; Papahadjopoulos et al., U.S. Pat. No. 4,235,871; Schneider, U.S. Pat. No. 4,224,179; Lenk, et al., U.S. Pat. No. 4,522,803; and Fountain, et al., U.S. Pat. No. 4,588,578. Popescu et al., U.S. Pat. No. 4,708,861 teaches sequestering liposomes in a gel matrix to control release of the active agent from the liposomes and protect the liposomes from dispersion and clearance by the body.

However, because liposomes themselves are degraded or cleared when administered in vivo, it is difficult to achieve prolonged release of a liposome-encapsulated agent in vivo. Moreover, liposomes are difficult to handle in terms of manufacture, sterilization, and storage.

Some researchers suggest using micelles for drug delivery. A micelle is an aggregate of surfactant molecules dispersed in a liquid colloid. A typical micelle in aqueous solution forms an aggregate with the hydrophilic “head” regions in contact with surrounding solvent, sequestering the hydrophobic tail regions in the micelle center. Kwon et al., U.S. Pat. No. 6,939,561 teaches methods for formulating hydrophobic therapeutic agents by incorporating them within micellular structures formed from block polymers comprising a hydrophilic backbone component, a spacer, and a hydrophobic core. Micelles are solid structures and are limited in the amount of active agent that they can hold, relying on the interaction of the active agent with the hydrophobic or hydrophilic portion to retain the active agent.

An issue with all of the above carriers for active agents is that release of the active agent is not controlled. It would be advantageous to have a drug delivery vehicle that protects the active agent from degradation until it is needed and then releases the active agent, preferably in response to a stimulus.

### SUMMARY OF THE INVENTION

The present invention is a drug delivery vehicle that includes active agent loaded vesicles in a hydrogel matrix. The vesicles are preferably made at least in part of a stimulus responsive polymer. The vehicle can be designed for the desired drug release profile. The vesicles are desirably designed to respond to a certain stimulus and the degree of the responsiveness can also be designed. Alternatively, the vesicles can be degradable, or can release active agent passively. The hydrogel matrix can also be selected to provide desired qualities. For example, the hydrogel matrix can be made at least partially of a responsive material so that it shrinks or expands in response to a stimulus. The vesicles can be released from an expanded hydrogel or the vesicles can be squeezed or pulled apart by a shrinking or expanding hydrogel.

The vehicle can be implanted or applied in situ to provide for the prolonged release of the encapsulated active agent. When administered in situ, the hydrogel matrix will desirably conform to the shape of the area where it is applied. In a preferred embodiment of the invention, the vehicle is applied topically, for example as a wound dressing. Other embodiments are oral and injectable drug delivery vehicles. In another embodiment of the present invention, the vehicle may be used as a support or overlay for cells grown in culture and thus provide for the prolonged release of the encapsulated agent into the culture medium.

In a preferred embodiment the vesicles are made from stimulus responsive amphiphilic copolymers.

As used herein, “drug” and “active agent” are synonymous. Examples include, but are not limited to, therapeutic, prophylactic, and diagnostic agents, as well as other materials such as cosmetic agents, fragrances, dyes, pigments, photoactive compounds, and chemical reagents, and other compounds with industrial significance. Active agents can also refer to metal particles, biological polymers, nanoparticles, biological organelles, and cell organelles.

“Stimulus” refers to an environmental characteristic such as, but not limited to, pH, temperature, light, ionic strength, electric field, magnetic field, and solvent composition. The term “stimulus” as used herein may refer to more than one stimulus.

“Responsive polymer” refers to a polymer having a physical change in response to a stimulus. These polymers have also been referred to as stimulus-responsive, environmentally sensitive, intelligent, or smart polymers.

“Responsive vesicle” or “responsive particle” refers to a vesicle having a permeability change in response to a stimulus.

“Hollow particle” and “vesicle” are synonymous and refer to a particle having a hollow core or a core filled with a material to be delivered or released. Vesicles may have a spherical or other shape.

“Encapsulation” refers to active agent contained in the vesicles, whether it is in the hollow center of the vesicles, in the membrane of the vesicles, or attached to the inside or outside of the vesicles.

“Free active agent” as used herein means active agent not encapsulated by a vesicle.

“Passive release” as used herein refers to release of active agent from a vesicle or a hydrogel matrix that is not in response to a stimulus.

“Active release” refers to release of active agent from a vesicle or hydrogel matrix upon a change in the vesicle or matrix. The change could be a response to a stimulus. The change could be due to degradation or a change in pore size due to swelling, for example.

### BRIEF DESRIPTION OF THE DRAWINGS

FIG. 1 is a schematic of a drug delivery vehicle of the invention.

### DETAILED DESCRIPTION OF THE INVENTION

Vesicles containing an active agent are entrapped in a hydrogel matrix to form a drug delivery vehicle. Release of the active agent can be modulated in several ways. Either or both of the vesicles and the hydrogel matrix can be manipulated to provide the release profile desired. In a desired embodiment, the vesicles are made at least in part of a stimulus responsive polymer; most desirably they are made at least in part of an amphiphilic stimulus responsive copolymer. In another embodiment, the active agent can be released from the vesicles in another way, such as through degradation of the vesicles or simple diffusion out of the vesicles.

Several embodiments of the vehicles are contemplated, wherein each of the vesicles and the matrix can be one or more of various types. The vesicles can be designed to respond to a certain stimulus and the degree of the responsiveness can be designed. More than one type of vesicle can be used in a vehicle, varying in degree of responsiveness (and speed of active agent release) and/or also in the type of stimuli to which they respond (temperature or pH, for example). Vesicles with different release rates (of the same active agent) and/or vesicles with different active agents can be included in the same vehicle.

The hydrogel matrix can be selected to also control the release of the active agent, by adjusting its degree of crosslinking or adding fillers, for example. The hydrogel matrix can simply allow the active agent to passively diffuse therethrough.

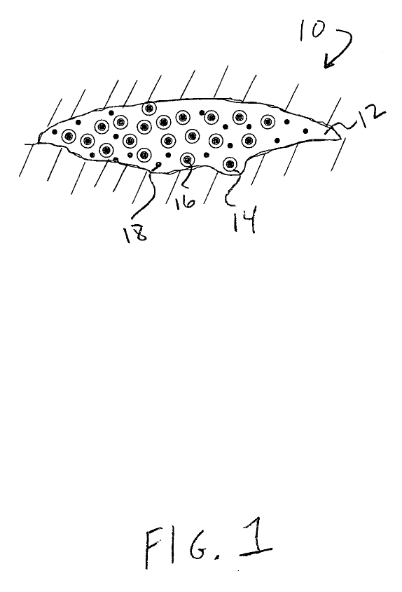

FIG. 1 illustrates one embodiment of the drug delivery vehicle 10 of the invention. A hydrogel matrix 12 encloses several vesicles 14. In this embodiment, active agent is both vesicle encapsulated active agent 16 and free active agent 18.

### The Vesicles

The vesicles encapsulate an active agent and release the active agent passively or actively. In a desired embodiment, the vesicles respond to a stimulus by undergoing a change in permeability to the active agent. The stimulus can be a change in any environmental factor such as, but not limited to, pH, temperature, light, ionic strength, electric field, magnetic field, and solvent composition. The change in permeability allows the active agent to be taken up by, or released from, the vesicle.

The vesicles preferably are made from, or include as a component, a responsive material. The responsive material is desirably a responsive polymer. The vesicles desirably have a shell made out of the responsive material, solely or in combination with other components.

An example of a pH responsive polymer that can be used is poly(acrylic acid) (PAAc). Hollow particles made out of PAAc change in size in response to a change in the pH of the solution. At a pH less than 5, the particles are in a compact, contracted state. The acrylic acid groups become increasingly dissociated with an increase in pH, leading to an increase in repulsive electrostatic interactions between the identically charged acrylate groups along the polymer backbone, which results in an expansion of the hollow particles. The particle radius increases from about 20 nm at a pH less than 4 to about 100 nm at a pH greater than 10. This corresponds to an increase of the enclosed volume by a factor of 125. The extent of this expansion depends, at a given pH and ionic strength, on the crosslinking density of the polymer network structure of the shell and on the presence of hydrophobic comonomers. Expansion of a particle results in an increase in its permeability to active agents below a certain size.

Other pH sensitive monomers include methacrylic acid (MAAc), maleic anhydride (MAnh), maleic acid (MAc), 2-acrylamido-2-methyl-1-propanesulfonic acid (AMPS), N-vinyl formamide (NVA), N-vinyl acetamide (NVA) (the last two may be hydrolyzed to polyvinylamine after polymerization), aminoethyl methacrylate (AEMA), phosphoryl ethyl acrylate (PEA), or methacrylate (PEMA). pH sensitive polymers may also be synthesized as polypeptides from amino acids (e.g. polylysine or polyglutamic acid) or derived from naturally occurring polymers such as proteins (e.g. lysozyme, albumin, casein, etc.), or polysaccharides (e.g. alginic acid, hyaluronic acid, carrageenan, chitosan, carboxymethyl cellulose, etc.) or nucleic acids, such as DNA. pH responsive polymers usually contain pendant pH sensitive groups such as ——PO(OH)₂, ——COOH, or ——NH₂ groups. With pH responsive polymers, small changes in pH can stimulate phase separation, similar to the effect of temperature on solutions of poly(N-isopropyl acrylamide (PNIPAM).

Analogous structural changes can be achieved in response to other stimuli, using particles made of appropriate stimulus responsive polymers. For example, a thermosensitive response may be observed for hollow particles of poly(N-isopropylacrylamide) (PNIPAM). Hydrophobic interactions in the neutral PNIPAM particles determine the swelling/deswelling behavior. This should lead to a considerable contraction of such particles with rising temperature. In this case charged comonomers can be used to influence the transition temperature and range.

By randomly copolymerizing a thermally sensitive N-isopropyl acrylamide (NIPAM) with a small amount (e.g. less than 10 mole percent) of a pH sensitive comonomer such as acrylic acid (AAc), a copolymer will display both temperature and pH sensitivity. Graft and block copolymers of pH and temperature sensitive monomers can be synthesized which retain both pH and temperature transitions independently.

Any of a number of stimulus responsive polymers can be used in the vesicles. Illustrative temperature, pH, ion, and/or light sensitive polymers are described by Hoffman, A. S., “Intelligent Polymers in Medicine and Biotechnology”, Artif Organs, 19, 458-467 (1995); Hoffman, A. S., “Intelligent Polymers in Medicine and Biotechnology”, Macromol. Symp., 98, 645-664 (1995); Chen, G. H. et al., “A new temperature- and pH-responsive copolymer for possible use in protein conjugation”, Macromol. Chem. Phys., 196, 1251-1259 (1995); Irie, M. et al., “Photoresponsive Polymers. Mechanochemistry of Polyacrylamide Gels having Triphenylmethane Leuco Derivatives”, Makromol. Chem., Rapid Commun., 5, 829-832 (1985); and Irie, M., “Light-induced Reversible Conformational Changes of Polymers in Solution and Gel Phase”, ACS Polym. Preprints, 27(2), 342-343 (1986).

The selection of monomers and control of molecular weight (by control of reactant concentrations and reaction conditions), structure (e.g. linear homopolymer, linear copolymer, block or graft copolymer, “comb” polymers, and “star” polymers), allow the design of polymers that respond to a specific stimulus and, in some embodiments, to two or more stimuli.

In a preferred embodiment, the hollow particles taught in U.S. Pat. No. 6,616,946 to Meier et al. are used (also referred to as particles or vesicles herein). These hollow particles are made of ABA or BAB triblock or AB diblock amphiphilic copolymers, containing one or more hydrophilic A blocks and one or more hydrophobic B blocks, that self-assemble in water to form hollow particles. A or B, or both, may be a stimulus responsive polymer. Alternatively, a stimulus responsive polymer may be mixed with the self-assembling polymers to form hollow particles, or after formation of the hollow particles. The stimulus responsive polymer may be entrapped within the particles at the time of formation, or chemically or ionically coupled to the amphiphilic polymers forming the self-assembling hollow particles. ABC tripolymers can also be used, where A and C are both hydrophilic or both hydrophobic, but are different polymers or the same polymer with different molecular weights.

The amphiphilic copolymers may be crosslinked or uncrosslinked. In one embodiment the triblock copolymers contain polymerizable end groups and/or side groups that are crosslinked by ionic, covalent, or other bonds to form hollow particles.

Accordingly, the amphiphilic segmented copolymers may consist in one embodiment of one segment A (hydrophilic) and one segment B (hydrophobic) (A-B-type, diblock). In another embodiment, the amphiphilic segmented copolymers may consist of one segment B and two segments A attached to its termini (A-B-A-type, triblock), or one of the hydrophilic blocks may be different, C (A-B-C type, triblock). In another embodiment, the amphiphilic segmented copolymers may have a comb-type structure wherein several segments A are pendent from one segment B, which may further carry one or two terminal segments A. Preferably, the copolymer is an ABA triblock copolymer.

Selection of the polymers, molecular weights, and other aspects of the hydrophobic and hydrophilic segments is covered in U.S. Pat. No. 6,616,946 to Meier et al. and one skilled in the art can look there and elsewhere for guidance. Preparation of the copolymers and the vesicles is also taught in the Meier patent and one skilled in the art can use the teachings therein to make the vesicles.

The copolymers, and thus the vesicles, may be degradable. One way to design degradable vesicles is by having the bond between the A and B or B and C segments degradable. Another way is to have either or both of A, B, or C degradable.

For polyelectrolytes, the pH and the pH interval necessary for the transition (i.e. the sharpness of the transition) can be systematically influenced using hydrophobic comonomers. Introducing n-butyl-methacrylate comonomers can shift the transition of poly(acrylic acid) hollow particles to higher pH values and, simultaneously, lead to a sharper (a first order-like) transition, occurring in a pH interval of only several tenths of pH units. Similar effects can be achieved with PNIPAM using charged comonomers.

Additionally, the surface of polymeric hollow particles can easily be modified with specific ligands. This can be achieved, for example, by copolymerization with a small fraction of ligand-bearing comonomers, e.g. galactosyl-monomers. It is well known that such polymer-bound galactosyl-groups are recognized by the receptors at the surface of hepatocytes (Weigel, et al. J. Biol. Chem. 1979, 254, 10830). Such labeled particles will diffuse or be released from the hydrogel and will migrate to and bind to the target.

The active agent can be trapped in the interior of the vesicle, or can be trapped in the membrane. More than one active agent can be encapsulated by the vesicle.

The hollow particles to typically range from about 50 nm to about 10 micrometers in diameter, although sizes may range from about 20 nm up to about 100 microns.

### The Hydrogel Matrix

The hydrogel matrix can be made of any of several types of biocompatible polymers. The polymer can be a synthetic or natural polymer. Representative synthetic polymers are: poly(hydroxy acids), polyanhydrides, polyorthoesters, polyamides, polycarbonates, polyalkylenes such as polyethylene and polypropylene, polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl acetate), polystyrene, polyurethanes and co-polymers thereof, derivativized celluloses, polymers of acrylic acid, methacrylic acid or copolymers or derivatives thereof, poly(butyric acid), poly(valeric acid), and poly(lactide-co-caprolactone), copolymers and blends thereof.

Examples of biodegradable polymers include polymers of hydroxy acids such as lactic acid and glycolic acid, and copolymers with PEG, polyanhydrides, poly(ortho)esters, polyurethanes, poly(butyric acid), poly(valeric acid), poly(lactide-co-caprolactone), blends and copolymers thereof.

Examples of natural polymers include proteins such as albumin and prolamines, for example, zein, and polysaccharides such as alginate, cellulose and polyhydroxyalkanoates, for example, polyhydroxybutyrate.

In a preferred embodiment, the hydrogel is the polymerizing hydrogel disclosed in U.S. Pat. No. 6,652,883 to Goupil et al. The degradable hydrogel disclosed in U.S. Pat. No. 6,710,126 to Hirt et al. could also be used. Other examples for a degradable hydrogel are taught in U.S. Pat. No. 5,986,043 and U.S. Publication No. 20060127352 to Hubbell et al. Non degradable hydrogels are taught in U.S. Pat. No. 5,932,674 to Mueller et al.

The hydrogel can function as a sieve for the vesicles, allowing their movement and release from the gel. The hydrogel can alternatively hold the vesicles in place and passively allow movement of the released active agent from the hydrogel.

The hydrogel can be degradable, either passively or in response to a stimulus.

In one embodiment, the hydrogel is an in situ polymerizing hydrogel, so that it is administered as a liquid and forms a gel in situ. Many examples of in situ polymerizing hydrogels can be found in the literature. In one preferred embodiment, the hydrogel matrix is a spray on wound dressing, such as disclosed in WO 03/063923 to BioCure, Inc.

### The Vehicles

Many different embodiments of the vehicles are possible. The hydrogel matrix encapsulates the vesicles but it can also include free active agent, which can be the same or different from the active agent in the vesicles. The vesicles and/or the free active agent can be released passively or actively from the vesicles and/or the matrix.

A passive matrix can be constructed from one or more hydrogel materials and can be constructed to have a desired pore size (also termed mesh size). Pore size can be adjusted using fillers and crosslinking. An active matrix can be constructed from a degradable hydrogel material, and can be programmed to degrade at a certain rate or in response to a certain pH, for example. An active matrix could be constructed from a hydrogel that is responsive, such as, for example, a hydrogel that swells or shrinks in response to a certain stimulus. Of course, the matrix could release one active agent passively and a different active agent actively. The matrix could release two different active agents, each in response to a different stimulus.

The vesicles can be of several designs, as discussed above. They can offer passive release of the active agent, or active release, via degradation or swelling, for example. Vesicles of different designs can be entrapped by a hydrogel matrix in a single drug delivery vehicle. These vesicles can contain different active agents and/or release the agent(s) with different release profiles.

In one embodiment, the vesicles release the active agent while the vesicles are entrapped by the hydrogel matrix; in another embodiment, the vesicles are released from the hydrogel matrix and then release the active agent. The hydrogel matrix can be designed to release the active agent or vesicles actively or passively.

The vesicles can be held by the hydrogel in any manner. For example, they can be crosslinked to the hydrogel or simply physically entrapped in the hydrogel.

The vehicle can be formulated in a number of ways. In one embodiment, a preformed embodiment, the vesicles containing the active agent and the hydrogel are mixed outside the body as the hydrogel forms, so that the vesicles are entrapped in the hydrogel matrix. This embodiment of the vehicle can be implanted or applied topically to an intended site. In other embodiments the vehicle is formed in situ, which offers the advantage of a vehicle that conforms to the shape of the intended area of application. Where the vehicle is formed in situ, such as by application of two parts that combine to form a hydrogel, the vesicles can be included in one or both of the pre-hydrogel parts. The vesicles are then entrapped in the hydrogel as it forms.

In one embodiment, the vehicle is formulated as a spray-on topical dressing, such as would be applied to a wound bed. Two parts that form a hydrogel when mixed together can be held in separate receptacles and then mixed together upon application. The vesicles can be in one or both receptacles, or in a separate receptacle. An initiator to formation of the hydrogel can be in one of the receptacles or applied separately.

### Active Agents

The vehicles are suitable for delivery of many types of active agents including therapeutic, diagnostic, or prophylactic agents as well as many compounds having cosmetic and industrial use, including dyes and pigments, fragrances, cosmetics, and inks. The agent is delivered to the target site where release occurs; for example as a function of the interaction of a stimulus with the stimulus-responsive material or as a function of simple degradation of the matrix or vesicle.

Both hydrophilic and hydrophobic drugs, and large and small molecular weight compounds, can be delivered. Drugs can be proteins or peptides, polysaccharides, lipids, nucleic acid molecules, or synthetic organic molecules. Examples of hydrophilic molecules include most proteins and polysaccharides. Examples of hydrophobic compounds include some chemotherapeutic agents such as cyclosporine and taxol. Agents that can be delivered include nucleic acids, pain medications, anti-infectives, hormones, chemotherapeutics, antibiotics, antivirals, antifungals, vasoactive compounds, immunomodulatory compounds, vaccines, local anesthetics, angiogenic and antiangiogenic agents, antibodies, anti-inflammatories, neurotransmitters, psychoactive drugs, drugs affecting reproductive organs, and antisense oligonucleotides. Diagnostic agents include gas, radiolabels, magnetic particles, radioopaque compounds, and other materials known to those skilled in the art.

Although described here primarily with reference to drugs, it should be understood that the vesicles can be used for delivery of a wide variety of agents, not just therapeutic or diagnostic agents. Examples include cosmetic agents, fragrances, dyes, pigments, photoactive compounds, and chemical reagents, and other materials requiring a controlled delivery system. Other examples include metal particles, biological polymers, nanoparticles, biological organelles, and cell organelles.

Large quantities of therapeutic substances can be incorporated into the central cavity of the vesicles. Active agents can be encapsulated into the polymer by different routes. In one method, the agent may be directly added to the copolymer during preparation of the copolymer. For example, the compound may be dissolved together with the polymer in ethanol. In a second method, the drug is incorporated into the copolymer after assembly and optionally covalent crosslinking. The hollow particles can be isolated from the aqueous solution and redissolved in a solvent such as ethanol. Ethanol is a good solvent for the hydrophilic and the hydrophobic parts of some polymers. Hence, the polymer shell of the hollow particles swells in ethanol and becomes permeable. Transferring the particles back into water decreases the permeability of the shell.

Vesicles that are made from a non-responsive polymer can be loaded through methods known to those skilled in the art, such as by contacting the vesicles with a solution of the active agent until the agent has been absorbed into the vesicles, the solvent exchange method or the rehydration method.

### EXAMPLES

### Example 1

### Passive Diffusion from Vesicles

The following vehicle will provide passive release of an active agent out of the vesicles and hydrogel matrix. Carboxy-fluorescein is soluble in both segments of the block copolymer and will slowly diffuse out of the vesicles and then be released from the hydrogel.

Vesicles are made out of the block copolymer poly(2-methyl-2-oxazoline)-b-polydimethylsiloxane-b-poly(2-methyl-2-oxazoline). Molecular weights of the segments are poly(2-methyl-2-oxazoline): 1300, and PDMS segment: 4400. The copolymer is made as described in U.S. Pat. No. 5,807,944 to Hirt et al.

A total of 50 mg of polymer was dissolved in 250 μl of ethanol (99%). The ethanolic solution was slowly added to 5 ml of bi-distilled water containing 0.2 M carboxy-fluorescein. A minimum of 4 h of stirring was allowed. Subsequently, the vesicles were extruded through 0.45 μm and 0.22 μm filters (Millex Durapore-PVDF, Millipore) 6 times. This procedure ensures formation of unilamellar vesicles with diameters dictated by the pore size of the extrusion filters and with a more monodisperse size distribution. Chromatographic separation was achieved on a Sepharose 4B column (1×30 cm) eluted with bi-distilled water. The slightly hazy fraction contains the vesicles.

The hydrogel matrix is based on a PVA-acrylamide macromer and a UV-initiator as taught by U.S. Pat. No. 7,070,809 to Goupil et al., Example 5a.

The resulting vesicles formulation is mixed with the PVA-acrylamide macromer to reach 7% solid and 0.5 wt % Irgacure 2959. The hydrogel is crosslinked with UV and immersed in a saline solution.

3 g of the vehicle are immersed in 5 ml of water. The water is exchanged every hour for the first 8 hours and every 24 hours thereafter and the fluorescence of the supernatant is measured in order to calculate the amount of carboxy-fluorescein released from the vehicle.

### Example 2

### Release from pH Responsive Vesicles

The following vehicle will provide release of an active agent out of the vesicles in response to a change in pH. The lidocaine HCl will be released slowly with increasing pH, with an increased rate after pH 5.

The vesicles are made from the block copolymer poly(2-vinylpyridine-b-ethylene oxide) (N_{P2VP}:29,N_{PEO}: 15) This polymer can be made as described in Foerster et al., Langmuir, 2006, 22, 5843-5847.

The hydrogel matrix is the same as Example 1.

The vesicles are loaded with lidocaine HCl, via a phase transfer method from chloroform to water, and cleaned over a Sepharose column. The resulting vesicles formulation is mixed with the PVA-acrylamide macromer and Irgacure 2959.3 g of the hydrogel is crosslinked with UV and immersed in 5 ml buffer solution at pH 4. The 5 ml buffer is exchanged after 8-16 h with 5 ml buffer solution at 0.5 pH units higher and left for another 8-16 h. This is repeated until pH 6.5 is reached. About 80% of free lidocaine is released from the hydrogel in 8 hours. The lidocaine HCl release is measured with UV at 263 nm and compared to a standard curve.

### Example 3

### Release by Hydrolysis

The following vehicle provides release of an active agent out of the vesicles as a result of hydrolysis of the vesicles.

The vesicles are made from the block copolymer polyethyleneglycol-b-polycaprolacton-b-polyethylenglycol (Mn: 1000-5000-1000) as taught in B. Jeong et al, Biomacromolecules 2005, 6, 885-890.

The hydrogel matrix is the same as in Example 1.

The vesicles are loaded with lidocaine HCl at pH 6.5 and cleaned over a Sepharose column as taught in Example 1. The resulting vesicles formulation is mixed with the PVA-acrylamide macromer and Irgacure 2959.5 times 3 g of the hydrogel is crosslinked with UV and immersed in 5 ml buffer solutions at different pH. The five solutions have pHs of 3, 4, 5, 6 and 7. The vesicles are left for 24 hours in the solution before it is exchanged for fresh buffer solution. The used buffer solution is analyzed by UV at 263 nm for the lidocaine concentration as taught in Example 2.

### Example 4

### Release from Temperature Sensitive Vesicles

The following vehicle provides release of an active agent out of the vesicles in response to a temperature change.

The vesicles are made from the block copolymer poly(2-methyl-2-oxazoline)-b-poly(2-isopropyl-2-oxazoline-co-2-butyl-2-oxazoline)-b-poly(2-methyl-2-oxazoline), where the ratio of isopropyl-oxazoline to butyloxazoline is 22:3, as taught in R. Jordan et al., Poly. Colloid Sci, 2008, Vol. 286, Number 4, 395-402.

The hydrogel matrix is the same as Example 1.

The vesicles are loaded with lidocaine HCl in a buffered solution at pH 6.5 above 40 C and cleaned over a Sepharose column. The resulting vesicles are mixed into 3 g of hydrogel at a solid content of 7% at 40 C. The hydrogel is cooled to room temperature and immersed in 10 ml of water. The vesicles will release their content slowly, due to the solubility of the poly(2-isopropyl-2-oxazoline-co-2-butyl-2-oxazoline) at room temperature in water. The release is measured with UV at 263 nm over 12 hours.

### Example 5

### Vesicles Crosslinked Into the Hydrogel

Vesicles are made from the block copolymer HO-poly(2-methyl-2-oxazoline)-b-polydimethylsiloxane-b-poly(2-methyl-2-oxazoline)-OH modified with isocyanto-ethylmetacrylate at the OH endgroups as taught in U.S. Pat. No. 5,807,944 to Hirt et al.

The hydrogel matrix is made as described in Example 1.

The vesicles are loaded with a hydrophilic lidocaine HCl and cleaned over a Sepharose column as taught above. The resulting vesicles formulation is mixed with the PVA-acrylamide macromer and Irgacure 2959.3 times 3 g of the hydrogel is crosslinked with UV and immersed in 10 ml buffer solution at pH 6.5 each. One sample is exposed to 50 C for 24 hours and left for another 24 hours. One sample is taken out of solution over night and put back into the buffer for 24 hours. Solutions are analyzed with UV for the lidocaine release and compared to the untreated solution. The shape change of the hydrogel due to the external influence has ruptured the vesicles and their content is released.

Modifications and variations of the present invention will be apparent to those skilled in the art from the forgoing detailed description. All modifications and variations are intended to be encompassed by the following claims. All publications, patents, and patent applications cited herein are hereby incorporated by reference in their entirety.

What is claimed is:

1. An active agent delivery vehicle comprising vesicles formed from self-assembled block copolymers and a hydrogel matrix, wherein the vesicles encapsulate an active agent and are entrapped in the hydrogel matrix.

2. The active agent delivery vehicle of claim 1, wherein the vesicles are made from at least one amphiphilic diblock (AB) or triblock (ABA or ABC) copolymer.

3. The active agent delivery vehicle of claim 1, wherein the vehicle releases the active agent with active release.

4. The active agent delivery vehicle of claim 3, wherein the vesicles release the active agent in response to a stimulus.

5. The active agent delivery vehicle of claim 1, wherein the vehicle releases the active agent by passive release.

6. The active agent delivery vehicle of claim 4, wherein the vesicles are pH sensitive and release the active agent upon a pH change.

7. The active agent delivery vehicle of claim 4, wherein the vesicles are temperature sensitive and release the active agent upon a change in temperature.

8. The active agent delivery vehicle of claim 4, wherein the vesicles are light sensitive and release the active agent upon exposure to light of a specific wave length.

9. The active agent delivery vehicle of claim 3, wherein the hydrophobic segment of the vesicle forming block copolymer in at least some of the vesicles is hydrolysable and these vesicles release the active agent after at least partial hydrolysis of the hydrophobic segment.

10. The active agent delivery vehicle of claim 4, wherein the vehicle contains vesicles having different responses to the stimulus and the vesicles release the active agent at different time points in order to extend the release time.

11. The active agent delivery vehicle of claim 4, wherein the vehicle contains vesicles having responses to different stimuli.

12. The active agent delivery vehicle of claim 9, wherein the vesicles are formed from of mixture of hydrolysable and non-hydrolysable block copolymers.

13. The active agent delivery vehicle of claim 1, wherein the hydrogel is an in situ polymerizable hydrogel and the vehicle is formed in situ in or on the body.

14. The active agent delivery vehicle of claim 1, wherein the hydrogel includes a free active agent.

15. The active agent delivery vehicle of claim 14, wherein the hydrogel releases the active agent by active release.

16. The active agent delivery vehicle of claim 3, where the hydrogel shrinks or expands upon a change in temperature or pH, which triggers the vesicles to release the active agent

17. The active agent delivery vehicle of claim 1, where the hydrogel is degradable in vivo.

18. A method for making an active agent delivery vehicle in situ in or on the body comprising the steps:
providing vesicles encapsulating an active agent;
providing an in situ polymerizing hydrogel;
mixing the vesicles and the in situ polymerizing hydrogel before or upon delivery of the in situ polymerizing hydrogel to the intended site of formation of the vehicle; and
delivering the mixed vesicles and in situ polymerizing hydrogel under conditions to polymerize the hydrogel.

19. The method of claim 18, where the vesicles include a polymer containing polymerizable endgroups and these polymerizable endgroups crosslink with the in situ polymerizing hydrogel.

20. The method of claim 19, where the polymerizable endgroups are acrylates, methacrylates, acrylamides, or styrene.

21. The method of claim 18, where the hydrogel is a two component system, the vesicles release active agent in response to a stimulus, the vesicles are in one component, and the second component contains a stimulus for the release of the active agent from the vesicles.
